(19) United States
(12) Patent Application Publication
(10) Pub. No.: US 2017/0253851 A1
(43) Pub. Date: Sep. 7, 2017

(54) NOVEL METHOD FOR CULTURE OF ALGAE, IN PARTICULAR MICROALGAE

(71) Applicant: FERMENTALG, Libourne (FR)

(72) Inventors: Pierre CALLEJA, LIBOURNE (FR); Marina LETTERIER, LIBOURNE (FR)

(21) Appl. No.: 15/506,372

(22) PCT Filed: Aug. 26, 2015
(86) PCT No.: PCT/FR2015/052273

§ 371 Date: Feb. 24, 2017

(30) Foreign Application Priority Data
Aug. 26, 2014 (FR) 1457999

(51) Int. Cl. C12N 1/12; C02F 3/34; C02F 3/32

(52) U.S. Cl. CPC C12N 1/12; C02F 3/322; C02F 3/34

(57) ABSTRACT

This relates to the field of culture of algae, in particular microalgae. Disclosed is a method for culture of algae, advantageously of microalgae. The particular culture conditions in terms of lighting and nutrients enables a biomass to be obtained which is constituted of microalgae having a quantity of chlorophyll that is at least low, advantageously very low, or even zero and a high quantity of antioxidant agents.

The present application relates to the field of culturing algae, particularly microalgae.

The invention particularly relates to a method for culturing algae, advantageously microalgae, characterized in that the specific culture conditions in terms of illumination and nutrients make it possible to obtain a biomass consisting of microalgae having a quantity of chlorophyll that is at least low, advantageously very low, and a high quantity of antioxidant agents.

Certain microalgae are mixotrophic beings, capable of both heterotrophy (absorption of organic matter in the culture medium) and autotrophy (use of light to capture carbon via photosynthesis). Mixotrophy is also called photoheterotrophy. The concept of mixotrophy extends to the use of light not only for photosynthesis but also as a light signal that can induce a metabolic response, for example the synthesis of pigments.

Heterotroph-dominant mixotrophy makes it possible to produce molecules of algal origin by coupling the advantages of both autotrophy and heterotrophy. It consists in introducing a light component of low intensity and short duration. As in heterotrophy, the organic substrate feeds the microalgae to produce large amounts of biomass but in this case the chloroplast and other light energy sensing organelles of the cell are activated.

These light energy sensors, or photoreceptors, are present in organelles such as the stigma which is regarded as a primitive eye that allows flagellated cells possessing one to move in relation to the quantity and the quality of the light. The other photosensitive organelles are the chloroplast, the chronoplast or the chromoplast.

These photoreceptors enable the cell to have higher productivity and to synthesize all the molecules that can be metabolized by a microalga. The molecules of interest produced by microalgae are of major industrial interest particularly in the fields of nutrition, cosmetics, green chemistry and energy.

These molecules are varied and include, for example, carbohydrates, proteins, amino acids, advantageously essential amino acids, and pigments, particularly photosynthetic pigments, the principal ones being chlorophylls, and carotenoids.

Several kinds of chlorophylls exist (chlorophylls a, b, c, d and f), which differ from one another by the details of their molecular structure and, further, by their specific absorption properties. Chlorophyll “a” is the most common photosynthetic pigment of the plant kingdom; it is found in all terrestrial and aquatic plants. Its light absorption peaks are at 430 nm and 660 nm. Chlorophyll “b” is found in microalgae but in a lesser amount. Its absorption peaks are centered at 450 nm and 645 nm.

According to the illumination parameters applied during a microalga culture, it is possible to direct the production of metabolites and particularly of pigments. The combination of a discontinuous light in the form of flashes and a specific light spectrum can be obtained by using light-emitting diodes (LEDs). The wavelengths can be selected in order to correspond to or to approach the absorption peak of carotenoids or chlorophylls. The induction of the accumulation of a pigment in particular can be obtained while limiting the accumulation of the other pigments present in the cell.

The term “carotenoid” encompasses carotenes (α, β, ε, γ, δ and ζ-carotene, lycopene and phytoene) and xanthophylls (astaxanthin, antheraxanthin, citranaxanthin, cryptoxanthin, canthaxanthin, diadinoxanthin, diatoxanthin, flavoxanthin, fucoxanthin, lutein, neoxanthin, rhodoxanthin, rubixanthin, siphonaxanthin, violaxanthin, zeaxanthin). Carotenoids are rather orange and yellow, liposoluble pigments. They are synthesized by all algae, all green plants and many fungi and bacteria (including cyanobacteria). They are absorbed by animals and human beings in their food. Carotenoids have two main absorption peaks located around 440 and 475 nm.

In plants, these are photosynthesis accessory pigments with two main roles: a light collecting role (transferring to the chlorophyll light energy that they absorb in the ranges of the spectrum located between violet and red) and a photoprotective role (collecting energy from the chlorophyll particularly in the case of excess light and shade), the presence of which enables the chlorophyll to avoid degradations due to photooxidation.

Carotenoids play an important role in nutrition and health, because several are provitamins A, and some also have anti-cancer and antioxidant activities. They also stimulate the synthesis of antibodies. Some of them are widely used in the food processing industry for their coloring properties, and also in the cosmetics and pharmaceuticals industries for their antioxidant properties and their photoprotective capacity.

Certain carotenoids are of great interest, such as lutein and astaxanthin, the latter particularly for its high antioxidant capacity.

Microalgae are of interest as an additional food source because they can be a source of proteins, fiber, lipids or antioxidant agents, particularly carotenoids. They can be used in native form, advantageously dried, but also processed, for example reduced in the form of flours.

They can be used in human food or animal feed, as a nutritional supplement, or incorporated in small amounts in foods, where they can replace eggs and fats, for example for bread-making.

Microalgae flours obtained from algae grown in open basins or in photobioreactors are available commercially.

There is a microalgae strain of the genus Chlorella (Chlorella protothecoides) that is included in the composition of many foods, because this strain has the distinctive characteristic of destroying its chlorophyll under heterotrophic conditions, i.e., when the medium contains a carbon source. Pigment-deficient mutant strains also exist (U.S. 2013/0122181).

Thus, except for certain mutants and certain wild algae strains such as those described above, the large majority of green microalgae have a high chlorophyll level, which makes them unappetizing due to their taste and their green color. Consequently, their use, as described above, is either limited or made complex if one wishes to reduce the quantity of chlorophyll in the biomass used in the desired application.

There thus remains a need for a microalgae biomass that is low in chlorophyll but rich in its other metabolites of interest which can be derived from the culture of microalgae strains that are common and commonly used in culture.

It would thus be advantageous to have a method for culturing “wild” microalgae (i.e., normally having in standard culture conditions a high chlorophyll level), a method which would make it possible to obtain a biomass of said microalgae that is low in chlorophyll but rich in its other metabolites of interest. Such a method should make it possible to reduce the amounts of chlorophyll pigments during the culture of wild microalgae, normally having in standard culture a high chlorophyll level.

The present invention aims to provide such a microalgae culture method, which can make it possible to better target the distribution of pigments in the microalgae produced, in order to obtain microalgae that are low in chlorophyll, while naturally maintaining a high level of antioxidant agents, advantageously carotenoids.

Such a biomass could then be used, as it is known, either in its native form, optionally dried, or in the form of a derivative such as for example in the form of flours, in all the known fields of application of microalgae or derivatives thereof (flours or others), particularly in nutrition, then impoverished in chlorophyll, said biomass or derivatives thereof then having a taste and a green color that are diminished, even non-existent, thus making the products including same more appetizing.

The inventors have shown, surprisingly and after long research, that culturing microalgae in a medium comprising at least one carbon source, and comprising at least one step of illumination with radiation having a narrow wavelength spectrum between 450 nm and 500 nm can make it possible to obtain a biomass that is low in chlorophyll and rich in antioxidant agents, particularly carotenoids. Indeed, the inventors have shown that illumination with radiation having a narrow spectrum centered at 475 nm wavelength will be absorbed by carotenoid pigments but absorbed very little by chlorophyll.

The culture method carried out with illumination of this type and with a culture medium comprising a carbon source makes it possible to obtain a biomass with a chlorophyll content lower than 500 ppm, even lower than 100 ppm, preferentially 20 or lower than 20 ppm, even more preferentially lower than 2 ppm, particularly in certain microalgae strains, while maintaining normal or even improved carotenoid production, i.e., a quantity of carotenoids between 50 and 10000 ppm, 1000 and 7500 ppm, advantageously between 2000 and 5000 ppm.

Microalgae, particularly certain strains, naturally produce lutein and, in a smaller amount, astaxanthin. It is possible to promote the production of astaxanthin relative to lutein by providing a high light intensity and/or by using a nitrogen-deficient medium (Cordero et al., Mar. Drugs 2012, 10, 2069-2088).

Advantageously, astaxanthin production can be improved relative to lutein production by providing a high light intensity and by using a nitrogen-deficient medium.

The method according to the invention makes it possible to increase the astaxanthin content in biomass obtained industrially. Moreover, the high light intensity can be limited to the selected wavelength (475 nm) to limit the production of chlorophyll.

According to the invention, “narrow” means that the light spectrum used is centered on a given wavelength and does not extend on each side of said wavelength more than 25 nm.

Thus, the invention has as a first object a method for culturing microalgae in a culture medium comprising at least one carbon source, and comprising at least one step of illumination with radiation having a narrow wavelength spectrum between 450 nm and 500 nm. Preferably according to the invention, the selected wavelength will be between 460 and 490 nm, most preferentially centered at 475 nm.

Illuminating the microalgae culture with light having a wavelength centered at 475 nm and whose spectrum does not extend beyond 25 nm on each side makes it possible to avoid the accumulation of chlorophyll by the microalgae with no other notable effect on the development of the microalgae in culture, particularly on the accumulation of other metabolites of interest contained in said microalgae, particularly antioxidant agents, most particularly carotenoids.

It is understood from the preceding that the invention consists of a variant of any known microalgae culture method, a variant that makes it possible on the basis of a known culture method to obtain a specific biomass. This variant (specific illumination conditions) can thus be included in any known microalgae culture method of the prior art.

Thus, according to the invention, the culture conditions for the algae strains can be the conditions known and used to cultivate the microalgae strains used. Advantageously, the conditions that will allow the best biomass yield will be used. The person skilled in the art will be able to integrate the illumination step according to the invention in a known method, in order to obtain the largest possible biomass, which meets the criteria according to the invention in terms of chlorophyll level and carotenoid level.

In this regard, mention may be made of the methods described by Cordero et al. (Marine Drugs, 2011, 9:1607-1624) and Bumbak et al. (Appl Microbiol Biotechnol, 2011, 91:31-46).

Favored methods are those allowing a high biomass yield. As an exemplary method, mention may be made of that described for example by Doucha and Livansky (J Appl Phycol, 2012, 24(1): 35-43).

More particularly, this step can be integrated in the methods described by the applicant in the applications No. WO2013/136027 and WO2012/035262.

According to the invention, the illumination can be produced by any means known to the person skilled in the art, in particular one or more lamps, one or more tubes, one or more light-emitting diodes (LEDs).

The inventors have shown that the method is even more effective when the illumination is provided by one or more light-emitting diodes (LEDs).

Thus, according to a variant of the invention, the illumination can be provided by one or more LEDs. The LEDs are preferably commercially available LEDs.

By way of example, mention may be made of LEDs from Seoul Optodevice Co., LTD (South Korea), from Nichia Corporation (Japan), or from SunLED Corporation (USA).

According to the method of the invention, said culture medium can be subjected to light radiation for a sufficient period corresponding at least to the period required for the desired criteria of chlorophyll level and carotenoid level to be met; this period will of course depend on the culture method in which this illumination step will be included. The person skilled in the art will be able without excessive experimentation to judge this required period. He will be able to adapt this time based on his knowledge of the field.

More particularly, mixotrophic conditions can be obtained under illumination conditions that are discontinuous and/or variable over time.

The term “discontinuous illumination” refers to illumination punctuated by periods of darkness. The illumination may in particular be in the form of flashes. A flash, within the meaning of the invention, is light illumination of given duration.

According to the invention, with regard to illumination, three concepts must be considered: the frequency or the number of flashes per unit of time, the duration of the flash and the intensity of the light emitted.

In terms of frequency, according to the invention, according to the number of flashes per unit of time used in the method according to the invention, two types of illumination are defined:

A low-frequency illumination wherein the number of flashes can be between about 2 and 3.6 10⁴ per hour (5.4·10⁻⁴ Hz to 10 Hz), preferentially between 3 and 3.6 10³ per hour (8.3·10⁻⁴ Hz to 1 Hz). It is understood here that the number of flashes per hour may have all the values between 2 and 36000 without it being necessary to mention them all (2, 3, 4, . . . , 35598, 35599, 36000).

A high-frequency illumination wherein the number of flashes can be between about 3.6×10⁴ and 5.4×10⁹ (10 Hz to 1.5·10⁶ Hz) per hour, preferentially between 3.6×10⁵ and 5.4×10⁹ (100 Hz to 1.5·10⁶ Hz). It is understood here that the number of flashes per hour may have all the values between 3.6×10⁵ and 5.4×10⁹ without it being necessary to mention them all (36000, 36001, 36002, . . . , 5399999998, 5399999999, 5400000000).

In terms of duration according to the invention, irrespective of the chosen illumination frequency, the flash duration can be between 1/150000 of a second and 1799 seconds (29 minutes and 59 seconds).

Of course, when high-frequency illumination is used, the flash duration can be preferentially between 1/150000 of a second and 1/10 of a second. And when low-frequency illumination is used, the flash duration can be preferentially between 1/10 of a second and 1799 seconds (29 minutes and 59 seconds).

In terms of light intensity according to the invention, the intensity of the light provided in the form of flashes can be between 5 and 5000 μmol. m⁻². s⁻¹, preferably between 5 and 2000 μmol. m⁻². s⁻¹, or 50 and 400 μmol. m⁻². s⁻¹, and more preferentially between 150 and 300 μmol. m⁻². s⁻¹ (1 μmol. m⁻². s⁻¹ corresponds to 1 μE m⁻². s⁻¹ (einstein), a unit often used in the literature).

According to the invention, the number of flashes per hour can be selected as a function of the intensity and the duration of the flashes (see above).

According to the invention, the concepts of frequency, duration and light intensity apply to the illumination as envisaged by the invention, i.e., the illumination produced by the chosen light source, advantageously by an LED, emitting a light radiation having a narrow spectrum between 450 and 500 nm, preferably centered at 475 nm and for the periods considered according to the invention.

According to another embodiment of the invention, the illumination can be variable, which means that the illumination is not interrupted by phases of darkness, but that the light intensity varies over time. This variation of light intensity is regular and can be periodic or cyclic. According to the invention, light may also be provided in a combination of continuous and discontinuous illumination phases.

The term “variable illumination” means that the light intensity varies in a regular manner at least twice per hour. An example of the illumination conditions suited to the method of invention is described in the international application of the Applicant published on 22 Mar. 2012 under No. WO2012035262.

The illumination may have, preferably, variations of intensity the amplitude of which generally is between 5 μmol. m⁻². s⁻¹ and 2000 μmol. m⁻². s⁻¹, preferably between 50 and 1500, more preferentially between 50 and 200 μmol. m⁻². s⁻¹.

According to a preferred embodiment, the illumination has variations of intensity the amplitude of which is between 5 and 1000 μmol.m⁻².s⁻¹, preferably between 5 and 400 μmol.m⁻².s⁻¹, these variations taking place between 2 and 3600, preferably between 2 and 200 times per hour.

These culture conditions make it possible to provide a defined quantity of light. This light provision may comprise discontinuous and/or variable illumination phases, with variations of intensity that may have identical or different amplitudes.

Advantageously according to the invention, the method may comprise, simultaneously or independently, any other step necessary to the growth of the biomass such as, for example, without being limited to, one or more culture step(s) without light or one or more biomass collection step(s).

The method according to the invention will of course ensue from the method chosen by the person skilled in the art for cultivating the chosen algae strain to which he will add one (or more) step(s) of illumination of the culture. The culture conditions will be thus dependent on the chosen method. In particular, the culture temperature will depend on the chosen strain.

Generally, the culture according to the method can be carried out at a temperature between 15° C. and 38° C., advantageously between 22° C. and 32° C.

According to the invention, the microalgae cultivated according to the method of the invention can be selected from all the strains of interest, particularly from the classes Chlorophyceae and Trebouxiophyceae, two classes of green algae which are phylogenetically very close (Lewis and McCourt, 2004 Am. J. Bot., 91 (10): 1535-1556). The classification of microalgae, originally based on morphological and biochemical characteristics, has been revised several times to integrate genetic data as it has become available. It turns out that certain genera like Chlorella and Chlamydomonas are polyphyletic, i.e., they are found in several classes. For example, members of the genus Chlorella are found in the classes Chlorophyceae and Trebouxiophyceae. Among the class Chlorophyceae, the chosen strains will belong advantageously to the genera Scenedesmus, Desmodesmus, Monoraphidium and Chlorella. Among the class Trebouxiophyceae, the chosen strains will belong advantageously to the genus Chlorella.

When the microalgae are of the genus Chlorella, they can be selected from the species C. acuminata, C. anitrata, C. antarctica, C. botryoides, C. conductrix, C. conglomerate, C. desiccate, C. emersonii, C. fusca, C. glucotropha, C. homosphaera, C. infusionum, C. luteoviridis, C. marina, C. miniata, C. minutissima, C. mirabilis, C. nocturne, C. oocystoides, C. ovalis, C. parasitica, C. parva, C. peruviana, C. protothecoides, C. pyrenoidosa, C. regularis, C. rugosa, C. saccharophila, C. saline, C. sorokiniana, C. spaerckii, C. stigmatophora, C. subsphaerica, C. variabilis, C. variegate, C. vulgaris, C. xanthella, C. zopfingiensis.

Advantageously according to the invention, the algae of the genus Chloralla can be algae selected from the species C. sorokiniana, C. vulgaris and C. saccharophila.

When the microalgae are of the genus Scenedesmus, they can be selected from the species S. abundans, S. aciculatus, S. aculeolatus, S. aculeotatus, S. acuminatus, S. acutiformis, S. acutus, S. aldavei, S. ambuehlii, S. anhuiensis, S. anomalus, S. apicaudatus, S. apiculatus, S. arcuatus, S. aristatus, S. armatus, S. arvernensis, S. bacillaris, S. baculiformis, S. bajacalifornicus, S. balatonicus, S. basiliensis, S. bernardii, S. bicaudatus, S. bicellularis, S. bidentatus, S. bijuga, S. bijugatus, S. bijugus, S. brasiliensis, S. breviaculeatus, S. brevispina, S. caribeanus, S. carinatus, S. caudato-aculeolatus, S. caudatus, S. chlorelloides, S. circumfusus, S. coalitus, S. costatogranulatus, S. crassidentatus, S. curvatus, S. decorus, S. denticulatus, S. deserticola, S. dileticus, S. dimorphus, S. disciformis, S. dispar, S. distentus, S. ecornis, S. ellipsoideus, S. ellipticus, S. falcatus, S. fenestratus, S. grahneisii, S. granulatus, S. gujaratensis, S. hanleyi, S. helveticus, S. heteracanthus, S. hindakii, S. hirsutus, S. hortobagyi, S. huangshanensis, S. hystrix, S. incrassatulus, S. indianensis, S. indicus, S. intermedius, S. jovais, S. jugalis, S. kerguelensis, S. kissii, S. lefevrei, S. littoralis, S. longispina, S. longus, S. mirus, S. multistriatus, S. naegelii, S. nanus, S. oahuensis, S. obliquus, S. obtusus, S. olvalternus, S. opoliensis, S. ovalternus, S. pannonicis, S. papillosum, S. parisiensis, S. parvus, S. pecsensis, S. perforatus, S. planctonicus, S. plarydiscus, S. platydiscus, S. pleiomorphus, S. polessicus, S. polyglobulus, S. polyspinosus, S. praetervisus, S. prismaticus, S. producto-capitatus, S. protuberans, S. pseudoarmatus, S. pyrus, S. quadrialatus, S. quadricauda, S. quadrispina, S. raciborskii, S. reginae, S. reniformis, S. rostrato-spinosus, S. rotundus, S. schnepfii, S. securiformis, S. semipulcher, S. senilis, S. serrato-perforatus, S. serratus, S. serrulatus, S. setiferus, S. smithii, S. soli, S. sooi, S. spinosus, S. spinulatus, S. striatus., S. subspicatus, S. tetradesmiformis, S. tricostatus, S. tschudyi, S. vacuolatus, S. velitaris, S. verrucosus, S. vesiculosus, S. weberi, S. wisconsinensis, S. wuhanensis, S. wuhuensis.

Advantageously according to the invention, the algae of the genus Scenedesmus can be algae selected from the species S. abundans, S. armatus and S. obliquus.

When the microalgae are of the genus Desmodesmus, they can be selected from the species D. abundans (in all its variations), D. aculeolatus, D. ambuehlii, D. armatus (in all its variations), D. arthrodesmiformis, D. asymmetricus, D. baconii, D. bicaudatus, D. bicellularis, D. brasiliensis (in all its variations), D. caudato-aculeatus (in all its variations), D. communis (in all its variations), D. costatogranulatus (in all its variations), D. cuneatus, D. curvatocornis, D. denticulatus (in all its variations), D. dispar, D. echinulatus, D. elegans, D. eupectinatus, D. fennicus, D. flavescens (in all its variations), D. gracilis, D. grahneisii, D. granulatus, D. hystricoides, D. hystrix, D. insignis, D. intermedius (in all its variations), D. itascaensis, D. kissii, D. komarekii (in all its variations), D. lefevrei (in all its variations), D. lunatus, D. magnus, D. maximus (in all its variations), D. microspina, D. multicauda, D. multiformis, D. multivariabilis (in all its variations), D. opoliensis (in all its variations), D. pannonicus, D. perdix, D. perforates (in all its variations), D. perforatus, D. pirkollei, D. pleiomorphus, D. polyspinosus, D. protuberans, D. pseudodenticulatus, D. pseudohystrix, D. pseudoserratus, D. quadricaudatus, D. regularis, D. santosii, D. schnepfii, D. serratoides, D. serrato-pectinatus, D. serratus, D. sp. CL1, D. sp. HegewalD. 1987-51, D. sp. Itas2/24S-1d, D. sp. Itas6/3T-2d, D. sp. Itas6/3T-2W, D. sp. Itas8/18S-6d, D. sp. Mary6/3T-2d, D. sp. NDem6/3P-3d, D. sp. NDem9/21T-10W, D. sp. Tow10/11T-12W, D. sp. Tow10/11T-17W, D. sp. Tow10/11T-1W, D. sp. Tow10/11T-2W, D. sp. Tow10/11T-3W, D. sp. Tow10/11T-6W, D. sp. Tow10/11T-8W, D. sp. Tow6/16T-10W, D. sp. Tow6/16T-15W, D. sp. Tow6/16T-16W, D. sp. Tow6/16T-17W, D. sp. Tow6/16T-26W, D. sp. Tow6/16T-31W, D. sp. Tow6/16T-32W., D. sp. Tow6/16T-35W, D. sp. Tow6/16T-8W, D. sp. Tow6/16T-9W, D. sp. Tow6/3T-11d, D. sp. Tow8/18P-13W, D. sp. Tow8/18P-14W, D. sp. Tow8/18P-1d, D. sp. Tow8/18P-25W, D. sp. Tow8/18P-3W, D. sp. Tow8/18P-4W, D. sp. Tow8/18T-10W, D. sp. Tow8/18T-23W, D. sp. Tow8/18T-25W, D. sp. Tow8/18T-5W, D. sp. WTwin8/18P-2d, D. spinosus, D. spinulatus, D. subspicatus (in all its variations), D. tropicus (in all its variations), D. ultrasquamatus.

Advantageously according to the invention, the algae of the genus Desmodesmus can be algae selected from the species D. istacaensis, D. intermedius and D. costado-granulatus.

When the microalgae are of the genus Monoraphidium, they can be selected from the species M. braunii, M. circinale, M. contortum, M. convolutum, M. dybowskii (in all its variations), M. griffithii, M. minutum, M. neglectum, M. pusillum, M. saxatile, M. terrrestre, M. sp. AKS-5, M. sp. Dek19, M. sp. FXY-10, M. sp. GK12, M. sp. IKA11, M. Itas (in all its variations), M. sp. KMMCC (in all its variations), M. sp. LUCC004, M. sp. No. 5F4, M. sp. NTAI03, M. sp. PTP1, M. sp. PTP4, M. sp. SS.

Advantageously according to the invention, the algae of the genus Monoraphidium can be algae selected from the species M. minutum, M. circinale and M. sp. GK12.

According to the invention, the culture method can be used to cultivate a single microalgae strain of a given genus, several strains of a single given genus, or several strains of different given genera (at least two species of two different genera).

According to the invention, the carbon source can be selected from any known and usable carbon source according to the chosen strain. The person skilled in the art will easily know how to choose the carbon source best suited to the strain to be cultivated. Examples of a usable carbon source include glucose, cellulose derivatives, lactate, starch, lactose, sucrose, acetate or glycerol.

The organic carbon substrate contained in the culture medium may consist of complex molecules or a mixture of substrates. Products derived from the biotransformation of starch, for example from corn, wheat or potato, in particular starch hydrolysates, which consist of small molecules, constitute, for example, organic carbon substrates suited to mixotrophic culture of the cells according to the invention.

The quantities of carbon sources used according to the method will of course depend on the chosen strain. Here again, the person skilled in the art will easily know how to adapt the quantities of carbon source to the strain to be cultivated in pure form or in mixture.

According to an embodiment of the invention, the organic carbon substrate may have a concentration between 5 mM and 1.5 M, preferably between 50 mM and 800 mM.

The culture can be carried out by any known culture technique, for example in flasks or in a reactor, but also in fermenters or in any container suited to microalgae growth such as, for example, raceway-type ponds, provided that said technique makes it possible contact the microalgae with at least the carbon source, and moreover is equipped with at least one light source emitting in the wavelengths having a narrow spectrum between 450 nm and 500 nm, preferably between 460 and 490 nm, most preferentially centered at 475 nm, the action of which on the culture will be able to lead to the desired biomass, i.e., a biomass that is low in chlorophyll and rich in antioxidant agents, particularly rich in carotenoids (carotenes and xanthophylls, particularly lutein and/or astaxanthin).

The method according to the invention may further comprise a step of collection of the microalgae. Said collection of the microalgae can be carried out by any technique enabling collection of the biomass, in particular gravimetric or low-pressure filtration methods, decantation methods, or even precipitation methods followed by gravimetric filtration.

According to a variant of the invention, the microalgae culture may also be subjected to a nitrogen deficiency and/or (simultaneously or independently) to a high light intensity.

By “nitrogen deficiency” is meant, according to the invention, a concentration of nitrogen (in any known form) in the culture medium between 0 and 0.5 mM, preferably between 0 and 100 μM.

Indeed, the inventors have shown that the culture of microalgae when at least the following combination of factors are combined:

presence of a carbon source;

subjection for a sufficient period to at least one illumination having a narrow wavelength spectrum between 450 nm and 500 nm;

low nitrogen concentration;

high light intensity;

leads to the production of a biomass not only low in chlorophyll and comprising a quantity of antioxidant agents, particularly carotenoids, that is at least normal or even improved, but also whose astaxanthin production is favored relative to lutein production.

Astaxanthin has a much higher oxidative capacity than lutein, stimulates the immune system, and has anti-inflammatory effects. It can thus be very advantageous to increase the quantity of astaxanthin, even if that supposes a lower quantity of lutein.

More precisely, according to this variant, the biomass thus produced has a lutein level between 10 and 1500 ppm, advantageously between 100 and 500 ppm and an astaxanthin level between 10 and 1000 ppm, advantageously between 200 and 500 ppm.

The invention also relates to the biomass that can be obtained by any one of the variants of the method according to the invention.

Microalgae have a high potential for use in many fields, examples of which include biofuel production, environmental remediation, particularly wastewater, human food or animal feed, cosmetics, medicine.

The biomass that can be obtained according to the method of the invention can obviously be used in all known fields of use of microalgae, particularly in food and/or cosmetics.

It is known that a microalgae biomass that can be obtained according to the invention can be used after harvest either directly, optionally dried, or after processing. In particular, microalgae biomasses are known to be used in the form of flours included in food compositions or in the form of food supplements.

The microalgae biomass that can be obtained according to the invention can be processed into flour according to any method known to the person skilled in the art. It can thus be envisaged, for example, that the microalgae can be separated from the culture medium, lysed and reduced to fine particles (average diameter of 10 microns), then dried.

The invention also relates to any use of the microalgae biomass that can be obtained according to the invention in any known field of use of microalgae, particularly biofuel production, environmental remediation, particularly wastewater, human food or animal feed, cosmetics, medicine, preferentially human food or animal feed, cosmetics.

The biomass obtained after culturing microalgae according to the method of the invention can make it possible to obtain in particular a flour that is low in chlorophyll (lower than 500 ppm, advantageously lower than 100 ppm, preferentially 20 or lower than 20 ppm, most advantageously lower than 2 ppm), rich in antioxidant agents, in particular carotenoids (carotenes and xanthophylls, particularly lutein and/or astaxanthin) in a quantity between 10 and 1000 ppm, 1000 and 7500 ppm, advantageously between 2000 and 5000 ppm, including in particular lutein in a quantity between 1000 and 10000, advantageously between 2000 and 5000, and/or astaxanthin in a quantity between 10 and 1000 ppm, advantageously between 200 and 500 ppm, meeting a need particularly in the food industry due to being more appetizing, having better taste, providing antioxidants in a large quantity and being able to be used in animal feed or human foods.

The invention thus relates to a flour that can be obtained after the processing of the microalgae biomass that can be obtained by the method according to the invention.

Irrespective of the form of use of the product that can be obtained by the method according to the invention (native or processed biomass), said product can be used pure or mixed with other ingredients traditionally used, particularly in food or cosmetics.

The invention also relates to any product that may comprise at least the algae biomass that can be obtained according to the invention.

The invention also relates to any product that may comprise at least the flour derived from the processing of the algae biomass that can be obtained according to the invention.

The following examples illustrate but do not however limit the present application.

### EXAMPLE 1: PRODUCTION OF CHLOROPHYLL PIGMENTS AND CAROTENOIDS BY THE MICROALGAE SCENEDESMUS AND DESMODESMUS

Scenedesmus abundans and Desmodesmus pannonicus cultures are prepared in 250 mL flasks with 50 mL of BG11 culture medium enriched in nitrogen (Rippka et al., 1979). The carbon substrate used is 10 g/L glucose. The culture temperature is set to 26° C.

The illumination conditions are as follows:

For heterotrophy: the cultures are maintained in darkness.

For mixotrophy: the cultures are illuminated in flashes (0.5 hertz, 1800 flashes per hour) with an intensity of 200 μmol.m⁻².s⁻¹. The light provision is obtained by LED lamps the wavelength of which extends from 450 nm to 500 nm, the peak being centered at 475 nm, for mixotrophy limited to blue light.

For “white” mixotrophy, the LEDs used cover a light spectrum from 370 to 700 nm, with 3 peaks centered at 445, 550 nm and 630 nm.

After 7 days of culture, the cells are centrifuged then lyophilized. Methods for extracting pigments are known to the person skilled in the art. The table below presents the results of 3 biological replicates.

| | Trophic | Chlorophyll a | Chlorophyll b | Lutein |
|---|---|---|---|---|
| Strain | mode | (ppm) | (ppm) | (ppm) |
| Scenedesmus | Heterotrophy | 99 (±40) | 58 (±29) | 68 (±27) |
| abundans | | | | |
| Scenedesmus | “Blue” | 210 (±31) | 304 (±58) | 3189 (±245) |
| abundans | mixotrophy | | | |
| | (450-500 nm) | | | |
| Scenedesmus | “White” | 7705 (±354) | 2101 (±287) | 3568 (±332) |
| abundans | mixotrophy | | | |
| Desmodesmus | Heterotrophy | 270 (±36) | 540 (±134) | 54 (±11) |
| pannonicus | | | | |
| Desmodesmus | “Blue” | 235 (±76) | 688 (±66) | 1857 (±163) |
| pannonicus | mixotrophy | | | |
| | (450-500 nm) | | | |
| Desmodesmus | “White” | 13886 (±204) | 4217 (±157) | 2289 (±141) |
| pannonicus | mixotrophy | | | |

The test carried out here shows the advantages of the method according to the invention. It is noted indeed that the “Blue” mixotrophy culture makes it is possible to obtain a biomass having a low chlorophyll a or b content relative to a “White” mixotrophy culture with a virtually identical but largely improved lutein level compared to a heterotrophic culture.

“Blue” mixotrophy combines the advantages of heterotrophy in terms of the quantity of chlorophyll a or b and of “White” mixotrophy in terms of lutein.

EXAMPLE 2: COMPARISON OF THE EFFECT OF ILLUMINATION (WAVELENGTH AND FLASHES) ON PIGMENT PRODUCTION BY CHLORELLA PROTOTHECOIDES UTEX B25

Composition of the medium:

| | | |
|---|---|---|
| Yeast extract | 4 | g/L |
| MgSO₄, 7H₂0 | 500 | mg/L |
| KH₂PO₄ | 1 | g/L |
| CaCl₂ | 44 | mg/L |
| Stock solution Fe—EDTA (6.9 g/L FeSO₄ and 9.3 g/L | 3 | mL/L |
| EDTA—Na₂) | | |
| Trace metal solution (3.09 g/L EDTA—Na₂; 0.080 g/L | 4 | mL/L |
| CuSO₄, 5H₂O; 2.860 g/L H₃BO₃; 0.040 g/L NaVO₃, | | |
| 4H₂O; 1.820 g/L MnCl₂; 0.040 g/L CoCl₂, 6H₂O; | | |
| 0.220 g/L; ZnSO₄, 7H₂O, 0.017 g/L Na₂SeO₃; 0.030 | | |
| g/L (NH₄)₆Mo₇O₂₄, 4H₂O) | | |
| Glucose | 30 | g/L |

Culture conditions:

In each Erlenmeyer flask 100 mL of 1% medium is inoculated with a 7-day-old Chlorella protothecoides preculture.

To test the effect of the light, the Erlenmeyer flasks are illuminated independently with a system of white LEDs or blue LEDs at 455 nm or at 475 nm. The light intensity for each condition is 0 μmol m⁻² s⁻¹ (μE) in heterotrophic conditions, 100 μmol m⁻² s⁻¹ with a frequency of 1 second of illumination every 10 seconds, and 5000 μmol m⁻² s⁻¹ with a frequency of 1 second of illumination every 60 seconds in mixotrophic conditions. The cells are cultivated at a temperature of 26° C. with moderate shaking (200 rpm). Cell growth is monitored every 24 hours by measuring absorbance at 800 nm. When the stationary phase is reached (7 days), 50 mL of cell suspension is taken to carry out the analysis of the quantities of pigments, chlorophyll and carotenoids contained in the biomass.

| | Carotenoids (ppm) | | | |
|---|---|---|---|---|
| | White | Blue light | Blue light | Hetero- |
| Light intensity and flash | light | (455 nm) | (475 nm) | trophy |
| 0 μE | — | — | — | 58 |
| 100 μE/1 sec every 10 s | 120 | 109 | 101 | — |
| 5000 μE/1 sec every 60 s | 180 | 134 | 162 | — |

| | Chlorophylls (ppm) | | | |
|---|---|---|---|---|
| | White | Blue light | Blue light | Hetero- |
| Light intensity and flash | light | (455 nm) | (475 nm) | trophy |
| 0 μE | — | — | — | 0 |
| 100 μE/1 sec every 10 s | 81 | 0 | 0 | — |
| 5000 μE/1 sec every 60 s | 200 | 106 | 20 | — |

The results show that in the absence of light (0 μE) there is an absence of chlorophyll and a presence of carotenoids. In the presence of low-intensity flashes (100 μE) the quantity of carotenoids in the biomass is higher than in heterotrophy irrespective of the wavelengths used. However, it can be noted that in blue light (455 nm or 475 nm) there is no chlorophyll production unlike that which is observed in white light. Flashes of very high intensity and low frequency seem to have an increased effect on carotenoid production compared to flashes of low intensity and high frequency. It should be noted that in the presence of white light and blue light at 455 nm the quantity of chlorophyll increases substantially by 200 and 106 ppm, respectively, whereas at 475 nm the quantity of chlorophyll is 10 to 5 times smaller.

1-27. (canceled)

28. Method for producing a biomass of microalgae consisting of microalgae having a lower quantity of chlorophyll and an increased quantity of antioxidant agents, comprising the following steps:
a) culturing the microalgae in a culture medium comprising at least one carbon source under conditions of illumination with radiation having a narrow wavelength spectrum between 460 and 490 nm and centered at 475 nm, and
b) harvesting the biomass of microalgae produced.

29. Method according to claim 28, wherein the illumination is produced by one or more lamp(s), one or more tube(s), one or more light-emitting diode(s) (LEDs).

30. Method according to claims 28 wherein the illumination is provided in the form of flashes with the number of flashes between 2 and 3.6 10⁴ per hour.

31. Method according to claim 28 wherein the illumination is provided in the form of flashes with the number of flashes between 3.6×10⁴ and 5.4×10⁹ per hour.

32. Method according to claim 30, wherein the flash duration is between 1/150000 of second and 1799 seconds (29 minutes and 59 sec).

33. Method according to claim 30, wherein the intensity of the light provided in the form of flashes is between 5 and 5000 μmol. m⁻². s⁻¹.

34. Method according to claim 28 wherein the illumination is not interrupted by phases of darkness and that the light intensity varies over time.

35. Method according to claim 34, wherein the variations of light intensity have an amplitude between 5 μmol. m⁻². s ⁻¹ and 2000 μmol. m⁻². s⁻¹.

36. Method according to claim 28 wherein the culture is carried out at a temperature between 15° C. and 38° C.

37. Method according to claim 28 wherein the microalgae is selected from the group consisting of microalgae of the genera Chlorella, Scenedesmus, Desmodesmus and Monoraphidium.

38. Method according to claim 28 wherein the carbon source is selected from the group consisting of glucose, cellulose derivatives, lactate, starch, lactose, sucrose, acetate and glycerol.

39. Method according to claim 28 wherein the carbon source is at a concentration between 5 mM and 1.5 M.

40. Method according to claim 28, wherein the biomass harvested has a chlorophyll content lower than 500 ppm.

41. Method according to claim 28, wherein the biomass harvested has a quantity of carotenoids between 50 and 10000 ppm.

42. Method according to claim 28 wherein the microalgae culture is subjected to a nitrogen deficiency.

43. Method according to claim 42, the nitrogen concentration in the culture medium is between 0 and 0.5 mM.

44. Method according to claim 31, wherein the flash duration is between 1/150000 of second and 1799 seconds (29 minutes and 59 sec).

45. Method according to claim 31, wherein the intensity of the light provided in the form of flashes is between 5 and 5000 μmol. m⁻². s⁻¹.
